(19) United States
(12) Patent Application Publication
(10) Pub. No.: US 2024/0397639 A1
(43) Pub. Date: Nov. 28, 2024

(54) WEARABLE ELECTRONIC DEVICE INCLUDING BATTERY

(71) Applicant: Samsung Electronics Co., Ltd., Suwon-si (KR)

(72) Inventors: Yongsub JEON, Suwon-si (KR); Gilho KIM, Suwon-si (KR); Hyejung KIM, Suwon-si (KR); Gijin PARK, Suwon-si (KR); Jooyil PYUN, Suwon-si (KR); Changryong HEO, Suwon-si (KR)

(21) Appl. No.: 18/647,645

(22) Filed: Apr. 26, 2024

Related U.S. Application Data
(63) Continuation of application No. PCT/KR2024/005193, filed on Apr. 18, 2024.

(30) Foreign Application Priority Data
May 26, 2023 (KR) 10-2023-0068288
Jun. 28, 2023 (KR) 10-2023-0083704

(51) Int. Cl. H05K 5/00

(52) U.S. Cl. CPC H05K 5/0086; H05K 5/0047

(57) ABSTRACT

According to various embodiments, a wearable electronic device may include: a first housing, a second housing coupled to an inner circumferential surface of the first housing, and at least one battery disposed between the first housing and the second housing, wherein the second housing includes a first molding layer disposed to be at least partially in contact with the at least one battery and having a specified compression rate, and a second molding layer configured to cover the first molding layer and coupled to the first housing.

### CROSS-REFERENCE TO RELATED APPLICATIONS

This application is a continuation of International Application No. PCT/KR2024/005193 designating the United States, filed on Apr. 18, 2024, in the Korean Intellectual Property Receiving Office and claiming priority to Korean Patent Application Nos. 10-2023-0068288, filed on May 26, 2023, and 10-2023-0083704, filed on Jun. 28, 2023, in the Korean Intellectual Property Office, the disclosures of each of which are incorporated by reference herein in their entireties.

### BACKGROUND

### Field

The disclosure relates to a wearable electronic device including a battery.

### Description of Related Art

An electronic device may include a wearable electronic device which is wearable on a part of a user's body to improve portability or user accessibility. A wearable electronic device may include a ring-type wearable device which provides various user experiences and useful functions by being worn on the user's finger. The wearable device may include at least one battery disposed therein, and a stable battery placement design may be required.

A wearable electronic device, especially a ring-type wearable electronic device worn on the user's finger, may be wirelessly connected to an external electronic device (e.g., smart phone). The wearable electronic device may acquire a user's biometric information via at least one sensor module and provide the acquired information to an external electronic device. In addition, the wearable electronic device may provide information provided from an external electronic device to the user visually, audibly, or tactilely. The wearable electronic device may include at least one battery disposed to provide power to electronic components arranged in an internal space. When the battery is used for a long period of time, a swelling phenomenon in which a certain area of the battery swells may occur.

However, an existing ring-type wearable electronic devices does not provide a structure which responds to the swelling phenomenon of a battery, and thus there is a problem in that a user may be exposed to danger while being attached to the human body, and the reliability of a product may also be reduced.

### SUMMARY

Embodiments of the disclosure may provide a wearable electronic device including a battery which may help ensure stability by providing a swelling space.

Embodiments of the disclosure may provide a wearable electronic device including a battery which may help maintain product reliability even when used for a long period of time.

The problems addressed by the disclosure are not limited to the above-mentioned problems, and may be expanded in various ways without departing from the spirit and scope of the disclosure.

According to various example embodiments, a wearable electronic device may include: a first housing, a second housing coupled to an inner circumferential surface of the first housing, and at least one battery disposed between the first housing and the second housing, wherein the second housing includes a first molding layer disposed to be at least partially in contact with the at least one battery and having a specified compression rate, and a second molding layer configured to cover the first molding layer and coupled to the first housing.

A wearable electronic device according to various example embodiments of the disclosure may include, as a second housing coupled to a first housing, a first molding layer disposed to surround at least a portion of a battery and having a predetermined compression rate, and a second molding layer configured to cover the first molding layer and coupled to the first housing, wherein the first molding layer accommodates a swelling portion of the battery caused by a swelling phenomenon of the battery, thereby helping ensure the reliability of the wearable electronic device.

In addition, various effects directly or indirectly identified via the disclosure may be provided.

The effects which can be obtained in the disclosure are not limited to those described above, and other effects not described above will be clearly understood by one of ordinary skill in the art to which the disclosure belongs based on the following description.

### BRIEF DESCRIPTION OF THE DRAWINGS

In relation to the description of drawings, the same or similar reference numerals may be used for the same or similar components. Further, the above and other aspects, features and advantages of certain embodiments of the present disclosure will be more apparent from the following detailed description, taken in conjunction with the accompanying drawings, in which:

FIG. 1 is a block diagram illustrating an example electronic device in a network environment according to various embodiments;

FIG. 2A is a perspective view of a wearable electronic device according to various embodiments;

FIG. 2B is a diagram illustrating a front view of the wearable electronic device of FIG. 2A according to various embodiments;

FIG. 3 is an exploded perspective view of a wearable electronic device according to various embodiments;

FIG. 4 is a cross-sectional view of the wearable electronic device taken along line 4-4 of FIG. 2A according to various embodiments;

FIGS. 5A, 5B, 5C and 5D are cross-sectional views of a wearable electronic device according to various embodiments;

FIG. 6 is a cross-sectional view of a wearable electronic device according to various embodiments; and

FIG. 7 is a flowchart illustrating an example assembly process of a wearable electronic device according to various embodiments.

### DETAILED DESCRIPTION

Hereinafter, with reference to the drawings, various example embodiments of the disclosure will be described in greater detail. However, the disclosure may be implemented in many different forms and is not limited to the embodiments described herein. In relation to the description of drawings, the same or similar reference numerals may be used for the same or similar components. In addition, in the drawings and related descriptions, descriptions of well-known functions and configurations may be omitted for clarity and brevity.

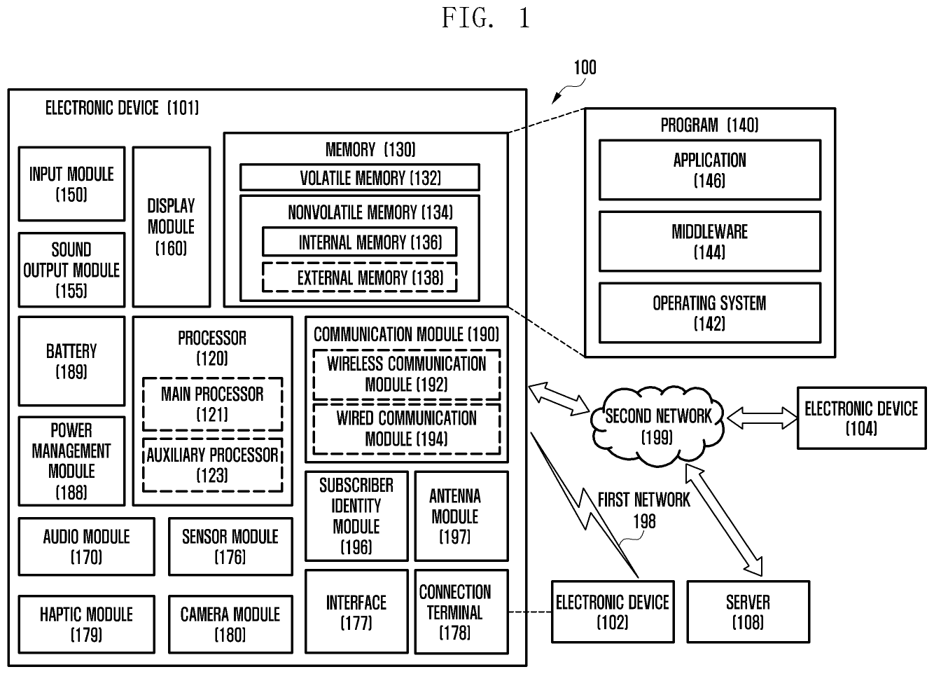

FIG. 1 is a block diagram illustrating an example electronic device in a network environment according to various embodiments.

Referring to FIG. 1, an electronic device 101 in a network environment 100 may communicate with an electronic device 102 via a first network 198 (e.g., a short-range wireless communication network), or an electronic device 104 or a server 108 via a second network 199 (e.g., a long-range wireless communication network). The electronic device 101 may communicate with the electronic device 104 via the server 108. The electronic device 101 includes a processor 120, memory 130, an input module 150, a sound output module 155, a display module 160, an audio module 170, a sensor module 176, an interface 177, a connection terminal 178, a haptic module 179, a camera module 180, a power management module 188, a battery 189, a communication module 190, a subscriber identification module (SIM) 196, or an antenna module 197. In various embodiments, at least one (e.g., the display device 160 or the camera module 180) of the components may be omitted from the electronic device 101, or one or more other components may be added in the electronic device 101. In various embodiments, some of the components may be implemented as single integrated circuitry. For example, the sensor module 176 (e.g., a fingerprint sensor, an iris sensor, or an illuminance sensor) may be implemented as embedded in the display device 160 (e.g., a display).

The processor 120 may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions. The processor 120 may execute, for example, software (e.g., a program 140) to control at least one other component (e.g., a hardware or software component) of the electronic device 101 coupled with the processor 120, and may perform various data processing or computation. As at least part of the data processing or computation, the processor 120 may load a command or data received from another component (e.g., the sensor module 176 or the communication module 190) in volatile memory 132, process the command or the data stored in the volatile memory 132, and store resulting data in non-volatile memory 134. The processor 120 may include a main processor 121 (e.g., a central processing unit (CPU) or an application processor (AP)), and an auxiliary processor 123 (e.g., a graphics processing unit (GPU), an image signal processor (ISP), a sensor hub processor, or a communication processor (CP)) that is operable independently from, or in conjunction with, the main processor 121. Additionally or alternatively, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display device 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). The auxiliary processor 123 (e.g., an ISP or a CP) may be implemented as part of another component (e.g., the camera module 180 or the communication module 190) functionally related to the auxiliary processor 123.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record, and the receiver may be used for an incoming calls. The receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. The display module 160 may include touch circuitry adapted to detect a touch, or sensor circuitry (e.g., a pressure sensor) adapted to measure the intensity of force incurred by the touch.

The audio module 170 may convert a sound into an electrical signal and vice versa. The audio module 170 may obtain the sound via the input device 150, or output the sound via the audio output device 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. The sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. The interface 177 may include, for example, a high definition multimedia interface (HDMI), a universal serial bus (USB) interface, a secure digital (SD) card interface, or an audio interface.

The connection terminal 178 may include a connector via which the electronic device 101 may be physically connected with the external electronic device (e.g., the electronic device 102). The connection terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. The haptic module 179 may include, for example, a motor, a piezoelectric element, or an electric stimulator.

The camera module 180 may capture an image or moving images. The camera module 180 may include one or more lenses, image sensors, image signal processors, or flashes.

The power management module 188 may manage power supplied to the electronic device 101. The power management module 188 may be implemented as at least part of, for example, a power management integrated circuit (PMIC).

The battery 189 may supply power to at least one component of the electronic device 101. The battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the AP) and supports a direct (e.g., wired) communication or a wireless communication. The communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a cellular network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the SIM 196.

The wireless communication module 192 may support a 5G network, after a 4G network, and next-generation communication technology, e.g., new radio (NR) access technology. The NR access technology may support enhanced mobile broadband (cMBB), massive machine type communications (mMTC), or ultra-reliable and low-latency communications (URLLC). The wireless communication module 192 may support a high-frequency band (e.g., the mm Wave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element including a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In an embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

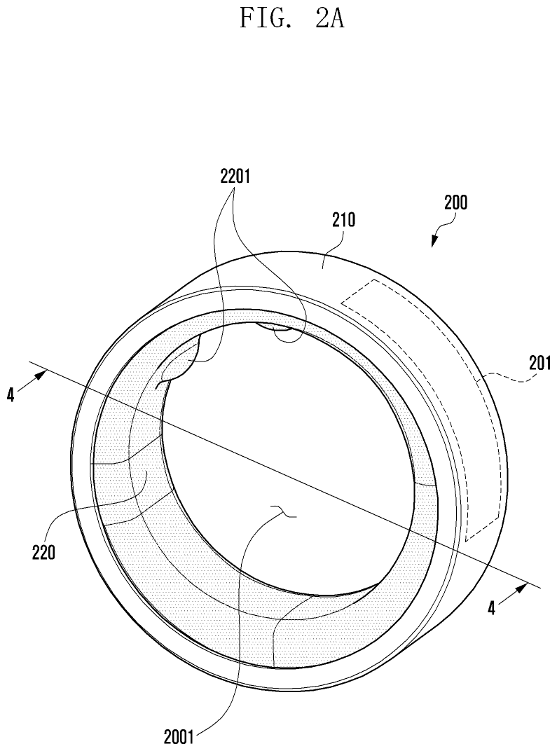

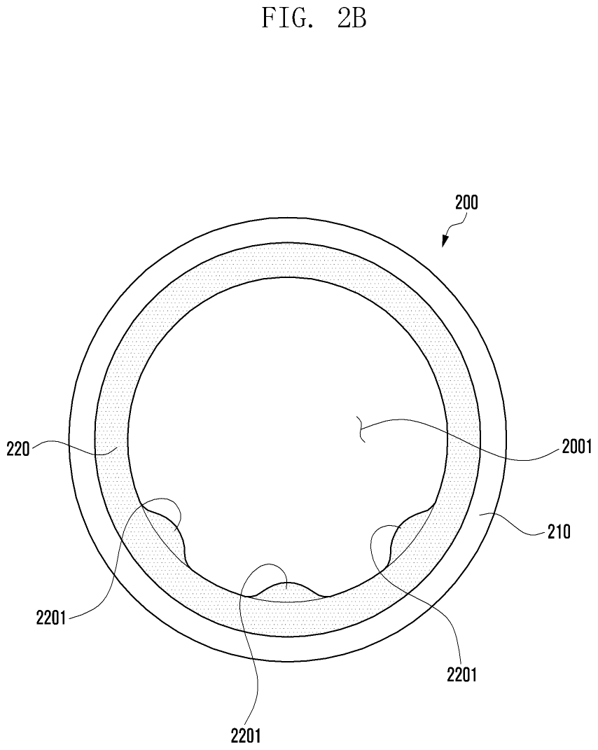

FIG. 2A is a perspective view of a wearable electronic device according to various embodiments. FIG. 2B is a diagram illustrating a front view of the wearable electronic device of FIG. 2A according to various embodiments.

A wearable electronic device 200 of FIGS. 2A and 2B may be similar to the electronic device 101 of FIG. 1 or may further include an embodiment of the electronic device.

In describing a wearable electronic device of the disclosure, a ring-type wearable electronic device 200 worn on a user's finger is shown and described, but is not limited thereto. For example, it will be apparent to one skilled in the art that example embodiments of the disclosure may be applied to a bracelet-type wearable electronic device, an open ring-type electronic device having an open portion, or a curved or non-curved electronic device.

Referring to FIGS. 2A and 2B, a wearable electronic device 200 may be formed in a ring shape including an opening 2001 therein. In an embodiment, the wearable electronic device 200 may include an annular first housing 210 (e.g., an external ring housing, a first ring housing, or a first housing portion) and an annular second housing 220 (e.g., an internal ring housing, a second ring housing, or a second housing portion) coupled to the first housing 210 and including the opening 2001. In an embodiment, the opening 2001 may be formed in a size to fit the user's finger. In an embodiment, the wearable electronic device 200 may include at least one protrusion 2201 protruding from the second housing 220 in the opening 2001 direction. In an embodiment, at least one protrusion 2201 may be disposed in the internal space of the wearable electronic device 200 and may have a protrusion amount and shape which are advantageous for detecting the external environment or contacting the user's skin. In an embodiment, at least one of the protrusions 2201 may be used by the user as a recognition member to prevent and/or reduce the wearable electronic device 200 from arbitrarily rotating on the user's finger.

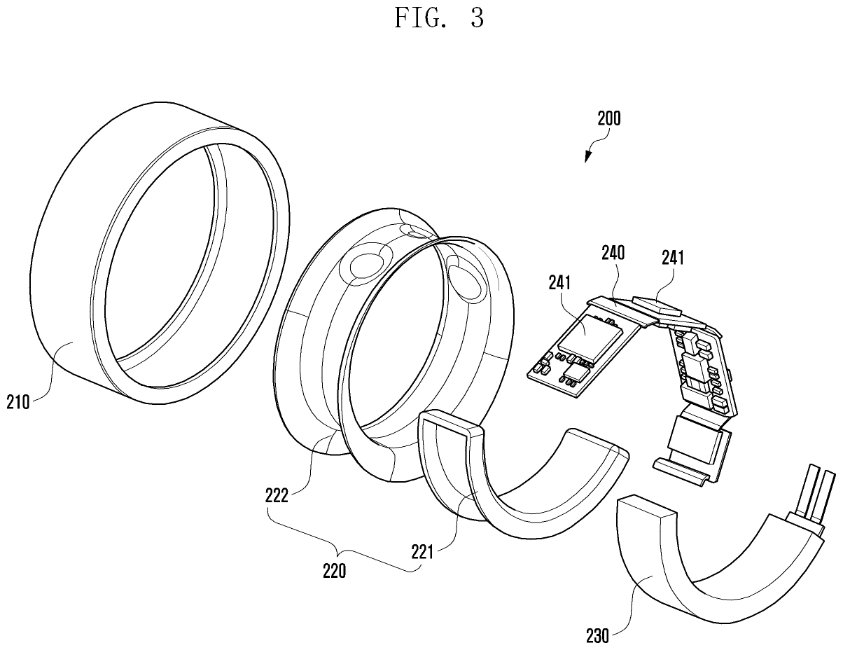

According to various embodiments, the wearable electronic device 200 may include at least one electrical element disposed in the space between the first housing 210 and the second housing 220. In an embodiment, the at least one electrical element may include at least one biometric sensor disposed to detect the user's biometric information via at least a portion of the second housing 220, a substrate (e.g., the substrate 240 in FIG. 3), a display 201, or an output module. In an embodiment, the substrate (e.g., the substrate 240 of FIG. 3) may include a flexible printed circuit board (FPCB) having flexibility to correspond to the curvature of the wearable electronic device 200. In an embodiment, the display 201 may be disposed to be visible from the outside through a portion (e.g., an outer circumferential surface) of the first housing 210. In an embodiment, the wearable electronic device 200 may further include an indicator such as an LED which may provide visual output information to the user, or may be replaced with the display 201. In an embodiment of the disclosure, the display 201 may be disposed in the form of a display which covers a portion or the whole area of the first housing 210 when viewed from the outside of the first housing 210. The area of the display 201 may be formed across all of the surface of the first housing 210. In an embodiment, the output module may include at least one speaker (not shown) to provide auditory output information to the user. In an embodiment, the output module may include a haptic module for providing tactile output information to the user.

According to various embodiments, a wearable electronic device 200 according to an example embodiment of the disclosure may be configured in such a way that a second housing 220 made of a polymer material is molded into a first housing 210 made of a hard material such as metal, ceramic, or polycarbonate (PC). In an embodiment, a curved battery disposed in the internal space of the wearable electronic device 200 (e.g., a battery 230 in FIG. 3) may swell in the direction of the second housing 220 formed by a molding method at the time of swelling. According to an embodiment of the disclosure, when a swelling phenomenon of the battery 230 occurs, the second housing 220 may be formed to guide the swelling direction of the battery. Therefore, due to the swelling phenomenon of the battery 230, the shape of the electronic device 200 may not be deformed and the battery may swell in the form of a pre-formed swelling guide in the second housing 220. Therefore, the wearable electronic device 200 according to an example embodiment of the disclosure may include a partial double molding structure in which a first molding layer (e.g., the first molding layer 221 in FIG. 3) is formed primarily to have a predetermined compression rate and a predetermined thickness to cover the battery (e.g., the battery 230 in FIG. 3), and a second molding layer (e.g., the second molding layer 222 of FIG. 3) formed on the first molding layer is coupled to the first housing 210, and thus the swollen portion via the swelling phenomenon of the battery (e.g., the battery 230 in FIG. 3) is accommodated in the first molding layer 221, thereby helping reduce damage to the wearable electronic device 200 and ensure the reliability of the electronic device.

FIG. 3 is an exploded perspective view of a wearable electronic device according to various embodiments.

Referring to FIG. 3, a wearable electronic device 200 may include a first housing 210, a second housing 220 coupled to the first housing 210, and a battery 230 disposed between the first housing 210 and the second housing 220. In an embodiment, the wearable electronic device 200 may include a substrate 240 disposed between the first housing 210 and the second housing 220 and including a plurality of electrical (electronic) elements 241. In an embodiment, the substrate 240 may include a flexible printed circuit board (FPCB) having flexibility to correspond to the curvature of the wearable electronic device 200. In an embodiment, the substrate 240 may include a substrate or a plurality of hard-type printed circuit boards (PCBs) including hard-type regions having a width and length which do not interfere with the curvature of the first housing 210 and/or the second housing 220. In an embodiment, the battery 230 may be disposed between the first housing 210 and the second housing 220 to be spaced apart from the substrate 240 at predetermined intervals, and may be electrically connected to the substrate 240 via a cable. In an embodiment, the battery 230 may be curved to have a curvature substantially the same as the curvature of the first housing 210. In an embodiment, the battery 230 may be disposed between the first housing 210 and the second housing 220 in a shape having a curvature different from the curvature of the first housing 210 and/or the curvature of the second housing 220.

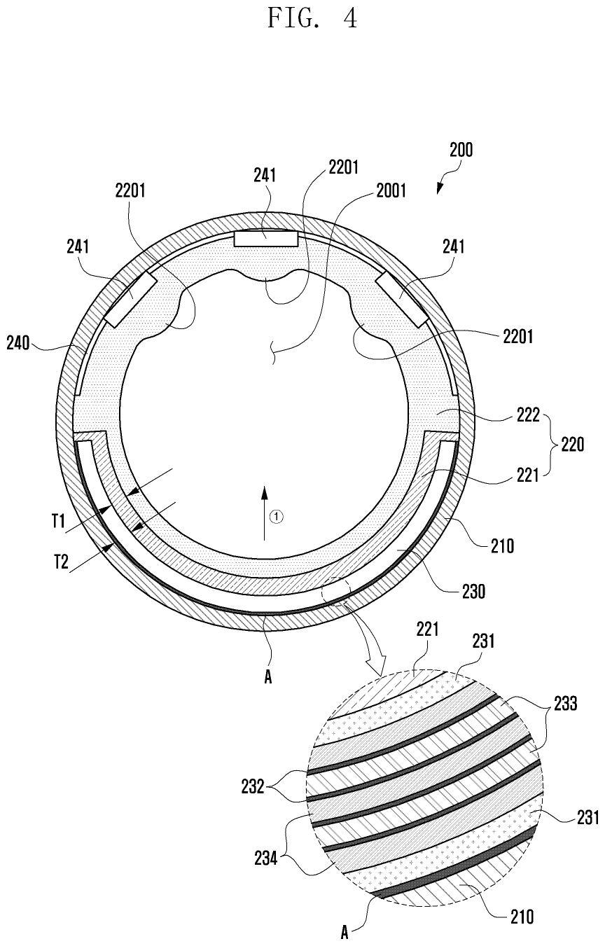

According to various embodiments, the first housing 210 may be formed of a metal material, ceramic, or a PC material. In an embodiment, the second housing 220 is a molding material and may be coupled to the first housing 210 via a molding process. In an embodiment, the second housing 220 may include a first molding layer 221 disposed to cover at least a portion of the battery 230, and a second molding layer 222 configured to cover the first molding layer 221 and coupled to the first housing 210. In an embodiment, the second housing 220 may further include an outer layer (not shown) disposed to surround a molding layer (e.g., the second molding layer 222). In an embodiment, the first molding layer 221 may be disposed to avoid being visible from the outside by the second molding layer 222. In an embodiment, the first molding layer 221 may be disposed to coverall of the battery 230 and to be at least partially in contact with the inner circumferential surface of the first housing 210. In an embodiment, the first molding layer 221 may be disposed adjacent to at least a portion of the battery 230 (e.g., at least a portion of the 1) direction surface of the battery in FIG. 4). In an embodiment, the first molding layer 221 may be formed of a polymer material having a predetermined compression rate. In an embodiment, the first molding layer 221 may be formed of polyurethane foam. In an embodiment, the compression rate of the first molding layer 221 may range from about 10% to about 50%. In an embodiment, the first molding layer 221 may include a flame-retardant in which a flame-retardant additive is added, the flame-retardant additive helping ensure the safety of a product by reducing the probability of fire spreading if the battery 230 is damaged or the battery ignites. According to an embodiment, the flame-retardant may include a halogen-based flame-retardant, a metal hydroxide flame-retardant, or a foamed flame-retardant. In an embodiment of the disclosure, the first molding layer 221 may include soft polyurethane foam which does not contain a flame-retardant additive. In an embodiment, polyurethane foam exhibits a wide range of stiffness, hardness, and density. Flexible polyurethane foam, which is a type of polyurethane foam, may be a material having predetermined elasticity. In an embodiment, the second molding layer 222 may be formed of epoxy which does not require compression rate. In an embodiment, the second molding layer 222 may be formed of a material having a compression rate lower than that of the first molding layer 211. According to an embodiment of the disclosure, the first molding layer 221 may be formed to have a thickness which does not exceed 200% of the maximum battery thickness.

FIG. 4 is a cross-sectional view of the wearable electronic device taken along line 4-4 of FIG. 2A according to various embodiments.

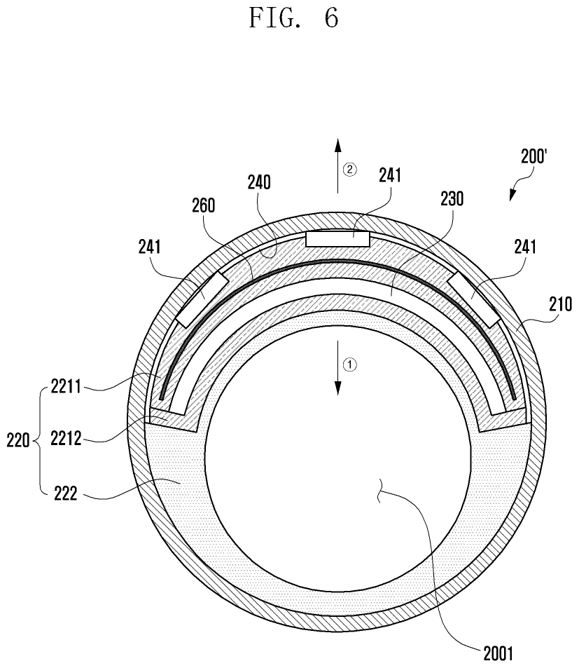

Referring to FIG. 4, a wearable electronic device 200 may include a first housing 210, a second housing 220 coupled to the first housing 210, a battery 230 disposed between the first housing 210 and the second housing 220, and a substrate 240 spaced apart from the battery 230 and including a plurality of electrical elements 241. In an embodiment, the battery 230 and the substrate 240 may be arranged in a direction facing each other. In an embodiment, the battery 230 and the substrate 240 may be arranged in a stacked structure overlapping with each other in a predetermined area of the electronic device 200 (e.g., the electronic device 200 of FIG. 6). In an embodiment, the second housing 220 may include a first molding layer 221 disposed to cover at least a portion of the battery 230 and a second molding layer 222 configured to cover the first molding layer 221 and coupled to the first housing 210. In an embodiment, the first molding layer 221 may be disposed to avoid being visible from the outside by the second molding layer 222. In an embodiment, the first molding layer 221 may be disposed to cover all of the battery 230 and to be at least partially in contact with the inner circumferential surface of the first housing 210. In an embodiment, the first molding layer 221 may be disposed to partially cover the battery 230, while mainly covering a swelling portion of the battery. In an embodiment, the battery 230 may be attached to the inner circumferential surface of the first housing 210 via a tape member (e.g., including an adhesive tape) A. In an embodiment, the battery 230 may be attached to the inner circumferential surface of the first housing 210 via bonding or ultrasonic fusion. In an embodiment, the thickness t1 of the first molding layer 221 may be determined in a range which does not exceed 200% of the battery thickness t2.

According to various embodiments, the battery 230 may include a plurality of base material portions 233 and 234 (e.g., current collectors) stacked in the internal space of a battery pouch 231. In an embodiment, the plurality of base material portions 233 and 234 may include positive electrode base material portions 233 and negative electrode base material portions 234. In an embodiment, the positive electrode base material portions 233 and the negative electrode base material portions 234 may be alternately stacked with each other via the separators 232 for electrical disconnection. In an embodiment, the positive electrode base material portions 233 and/or the negative electrode base material portions 234 may be formed of a highly conductive, bendable sheet-shaped metal material such as aluminum, stainless steel, nickel, titanium, or calcined carbon. Although not shown, the plurality of base material portions 233 and 234 may each include mixture layers (e.g., a positive electrode active material and a negative electrode active material) applied to one or both sides. In an embodiment, the mixture layers may be formed of a mixture of an active material, a conductive material, and a binder or the mixture with a filler added thereto. In an embodiment, separators 232 may include non-woven fabrics or sheets formed of olefin-based polymers such as chemical-resistant and hydrophobic polypropylene, glass fibers, or polyethylene. In an embodiment, the battery pouch 231 may include a hard-type PC having a predetermined curved shape. In an embodiment, the battery pouch 231 may be a film type and may be formed to be adaptively bendable to the curvature of the inner circumferential surface of the first housing 210.

According to various embodiments, when the battery 230 may be used for a long period of time, the electrolyte (e.g., lithium ion electrolyte) contained in the battery pouch 231 may vaporize and at least partially swell. For example, the battery 230 may be induced to swell only in the direction (e.g., direction \textcircled{1}) of the second housing 220 formed of a molding material, based on the first housing 210 formed of a hard-type material. In this case, the swelling portion of the battery 230 may be accommodated in the first molding layer 221 having a predetermined compression rate. Therefore, even if the battery 230 swells due to the swelling phenomenon, the battery may be accommodated in the first molding layer 221, and thus by reducing the risk of arbitrary separation or deformation of the first housing 210 and the second housing 220 due to the swelling pressure of the battery 230, the disclosure may help improve reliability by, for example, preventing and/or reducing deformation of the mechanism structure due to battery swelling of the wearable electronic device 200.

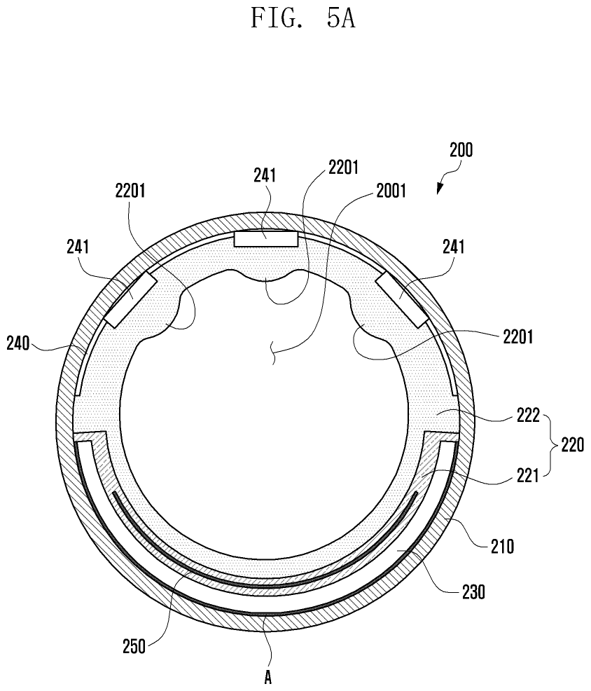

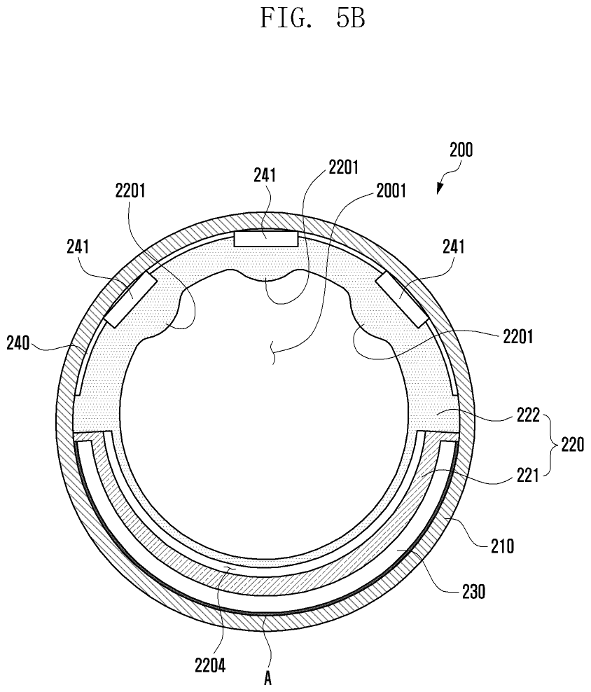

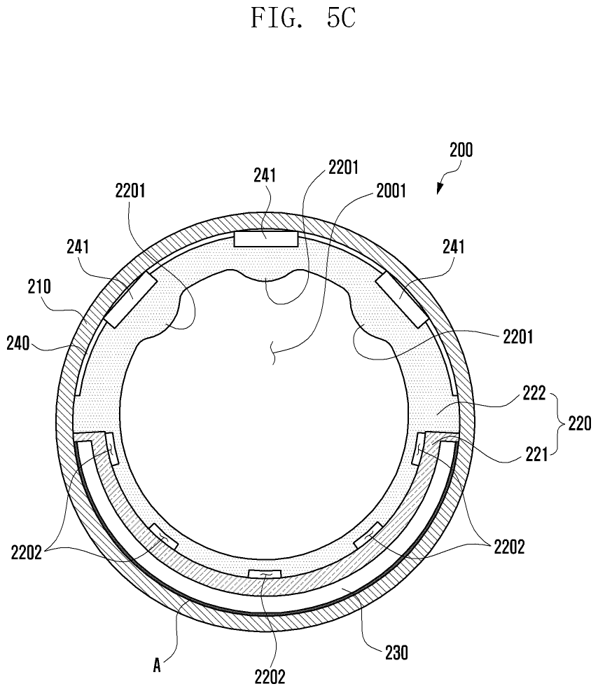

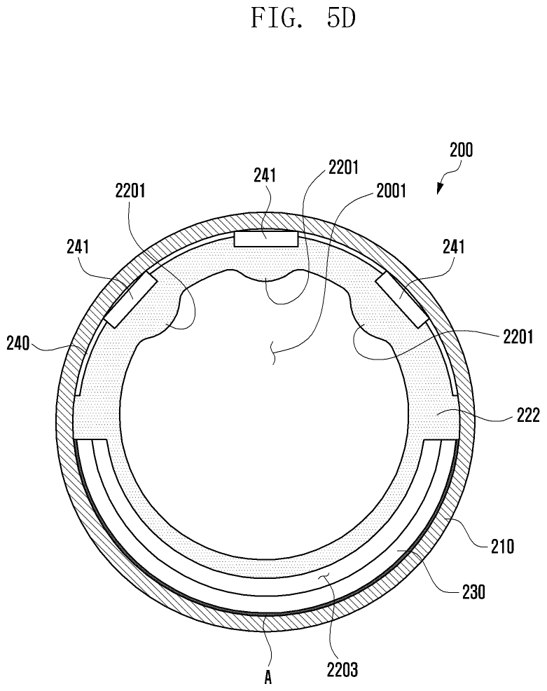

FIGS. 5A, 5B, 5C and 5D are cross-sectional views of a wearable electronic device according to various embodiments.

In describing a wearable electronic device 200 of FIGS. 5A, 5B, 5C and 5D (which may be referred to as FIGS. 5A to 5D), the components substantially the same as those of the wearable electronic device 200 of FIG. 4 are given the same reference numerals, and the detailed description thereof may not be repeated here.

Referring to FIG. 5A, the wearable electronic device 200 may further include a support member (e.g., a support or plate) 250 accommodated inside the first molding layer 221. In an embodiment, the support member 250 may be formed of a bendable plate-shaped metal material (e.g., stainless steel) to help support a portion which swells due to swelling of the battery 230 or to help reduce the user's risk exposure due to damage such as an explosion. In an embodiment, the support member 250 may be embedded internally when forming the first molding layer 221. In an embodiment, the support member 250 may be disposed at the boundary between the first molding layer 221 and the second molding layer 222. In this case, the support member 250 may be disposed to be attached to the outer surface of the first molding layer 221. In an embodiment, the support member 250 may be disposed between the battery 230 and the first molding layer 221. In this case, the support member 250 may be disposed to be attached to the outer surface of the battery 230. For example, the support member 250 may be formed of a bendable plate-shaped metal material, may thus be deformed to respond to the swelling portion caused by the swelling phenomenon of the battery 230, but may effectively protect against damage such as explosion. In an embodiment, a plurality of support members 250 may be arranged. In an embodiment, when swelling of the battery 230 occurs, in order to guide the battery 230 in a predetermined shape, the plurality of support members 250 may be formed in a specific area to at least partially overlap with each other or to be spaced apart from each other at designated intervals.

Referring to FIG. 5B, the wearable electronic device 200 may include a cavity 2204 disposed between the first molding layer 221 and the second molding layer 222 in at least a portion of the area corresponding to the battery 230. In an embodiment, the cavity 2204 may be formed by changing the shape of the first molding layer 221 and/or by changing the shape of the second molding layer 222. In an embodiment, the portion of the battery 230 which swells via the swelling phenomenon may be more flexibly received in the cavity 2204, so that the pressure applied to the second molding layer 222 may be distributed or reduced.

Referring to FIG. 5C, the cavity 2204 in FIG. 5B may be replaced with a plurality of cavities 2202 spaced apart from each other at designated intervals at the corresponding locations.

Referring to FIG. 5D, the portion of the battery 230 which swells via the swelling phenomenon may be accommodated in a cavity 2203 formed to be spaced a designated interval apart from the battery 230 via the second molding layer 222 without the first molding layer 221. In an embodiment, the wearable electronic device 200 may further include a support member (not shown) accommodated inside the second molding layer 222. In an embodiment, the support member may be formed of a bendable plate-shaped metal material (e.g., stainless steel) and may thus help support a portion which swells due to the swelling phenomenon of the battery 230 or reduce the user's risk exposure due to damage such as an explosion.

FIG. 6 is a cross-sectional view of a wearable electronic device according to various embodiments.

In describing a wearable electronic device 200′ of FIG. 6, the components substantially the same as those of the wearable electronic device 200 of FIG. 4 are given the same reference numerals, and the detailed description thereof may be omitted.

Referring to FIG. 6, a wearable electronic device 200′ may include a first housing, a second housing 220 coupled to the first housing 210, a battery 230 disposed between the first housing 210 and the second housing 220, and a substrate 240 disposed to overlap with the battery 230 and including a plurality of electrical elements 241. In an embodiment, the substrate 240 may be disposed to be attached to the inner circumferential surface of the first housing 210, and the battery 230 may be disposed between the substrate 240 and the second molding layer 222 by soft molding layers 2211 and 2212 (e.g., the first molding layer 221 in FIG. 4). In an embodiment, the soft molding layers 2211 and 2212 may include a first soft molding layer 2211 disposed to cover the substrate 240, and a second soft molding layer 2212 disposed to cover the battery 230 disposed on the first soft molding layer 2211. In this case, after the first soft molding layer 2211 is cured, the battery 230 may be disposed to be attached to the cured first soft molding layer 2211, and may be fixed via the second soft molding layer 2212 disposed to cover the battery 230 and the cured first soft molding layer 2211. In an embodiment, the second soft molding layer 2212 may be disposed to at least partially or totally cover the battery 230 and to partially cover the first soft molding layer 2211. The first soft molding layer 2211 and the second soft molding layer 2212 may be formed of polyurethane foam having a predetermined compression rate. In an embodiment, the second molding layer 222 may be coupled to the first housing 210 to cover both the first soft molding layer 2211 and the second soft molding layer 2212. In this case, the first soft molding layer 2211 and the second soft molding layer 2212 may be arranged to avoid being visible from the outside. In an embodiment, the wearable electronic device 200′ may include a heat dissipation member 260 (e.g., a graphite or metal sheet) disposed between the substrate 240 and the battery 230, inside the first soft molding layer 2211. For example, the heat dissipation member 260 may reduce the amount of heat produced from the substrate 240 or the battery 230 and transferred to the battery 230 or the substrate 240.

According to various embodiments, the battery 230 may swell in the opening direction (e.g., \textcircled{1} direction) and the opposite direction (e.g., direction \textcircled{2} via the swelling phenomenon. In this case, the swelling portion of the battery 230 may be accommodated in the first soft molding layer 2211 and the second soft molding layer 2212 having a predetermined compression rate, and thus by reducing the risk of arbitrary separation or deformation of the first housing 210 and the second molding layer 222 due to the swelling pressure of the battery 230, the disclosure may help improve the reliability of the wearable electronic device 200′.

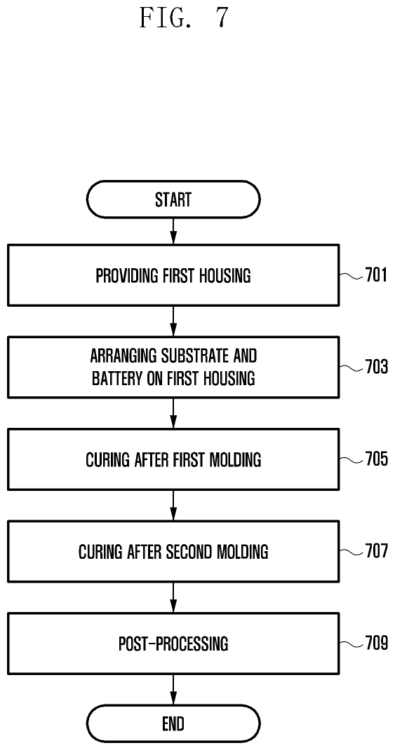

FIG. 7 is a flowchart illustrating an example assembly process of a wearable electronic device according to various embodiments.

Referring to FIG. 7 and the above-described drawings, in operation 701, a first housing 210 may be provided. In an embodiment, the first housing 210 may be formed in an annular shape. (However, the shape is not limited to an annular shape and may be applied even to a non-annular shape. For example, the exterior (the first housing 210) may have a square or non-square shape, and the interior (the second housing 220) may have an annular shape.) In an embodiment, the first housing 210 may be formed of metal, ceramic, or PC. In an embodiment, the first housing 210 may be formed to include an accommodation portion for arranging various electrical elements such as the battery 230 and/or the substrate 240.

In operation 703, the battery 230 and the substrate 240 may be arranged on the first housing 210. In an embodiment, the substrate 240 may be a flexible substrate and may be disposed to be attached to the inner circumferential surface of the first housing 210. In an embodiment, the battery 230 may also be disposed in a position different from that of the substrate 240 by being attached to the inner circumferential surface of the first housing 210 via an adhesive member A or the like.

In operation 705, a molding material (e.g., polyurethane foam) having a predetermined compression rate may be applied and molded to cover the battery 230, and then the first molding layer 221, which is cured via a vacuum defoaming process, may be formed. In an embodiment, the coated molding material may be cured via heat or ultraviolet rays. In an embodiment, the coated molding material may be cured via a natural curing process.

In operation 707, a molding material (e.g., epoxy) is applied and molded to cover the first molding layer 221 and to be coupled to the first housing 210, and then the second molding layer 222 cured via a vacuum defoaming process may be formed. In an embodiment, the first molding layer 221 may be disposed to avoid being visible from the outside via the second molding layer 222. In an embodiment, the first molding layer 221 and the second molding layer 222 may be the second housing 220 coupled to the first housing 210. In an embodiment, when the battery 230 and the substrate 240 are arranged to overlap with each other, as in the case of FIG. 6, three molding processes may be performed individually and sequentially when forming each molding layer (e.g., the first soft molding layer 2211, the second soft molding layer 2212, and the second molding layer 222).

In operation 709, the first housing 210 and the second molding layer 222 coupled to the first housing 210 may be post-processed. For example, the post-treatment process may include removal of residues remaining after the molding process, or, if necessary, at least partially performed polishing, shape processing, or painting.

According to various example embodiments, a wearable electronic device (e.g., the wearable electronic device 200 of FIG. 4) may include: a first housing (e.g., the first housing 210 of FIG. 4), a second housing (e.g., the second housing 220 of FIG. 4) coupled to the inner circumferential surface of the first housing, and at least one battery (e.g., the battery 230 of FIG. 4) disposed between the first housing and the second housing, wherein the second housing includes a first molding layer (e.g., the first molding layer 221 of FIG. 4) disposed to be at least partially in contact with the at least one battery and having a specified compression rate, and a second molding layer (e.g., the second molding layer 222 of FIG. 4) configured to cover the first molding layer and coupled to the first housing.

According to various example embodiments, the first molding layer may be disposed to avoid being visible from the outside.

According to various example embodiments, the first molding layer may be disposed to overlap the battery to cover all of the battery.

According to various example embodiments, the battery may have a curvature corresponding to that of the first housing.

According to various example embodiments, the battery may be attached to at least a portion of the first housing via an adhesive member comprising an adhesive tape.

According to various example embodiments, the first housing or the second housing may have an annular shape.

According to various example embodiments, the wearable electronic device may include a support member (e.g., the support member 250 of FIG. 5A) comprising a support or plate disposed between the battery and the second molding layer.

According to various example embodiments, the support member may include a plate-shaped metal material disposed inside the first molding layer.

According to various example embodiments, the support member may be disposed to be attached to the outer surface of the first molding layer between the first molding layer and the second molding layer.

According to various example embodiments, the wearable electronic device may include at least one cavity (e.g., the cavity 2204 of FIG. 5B or the cavities 2202 of FIG. 5C) disposed between the first molding layer and the second molding layer.

According to various example embodiments, the wearable electronic device may include a substrate (e.g., the substrate 240 of FIG. 4) disposed between the first housing and the second housing and including at least one electronic element (e.g., the electrical elements 241 of FIG. 4), wherein the substrate is spaced apart from the battery.

According to various example embodiments, the substrate may include a flexible printed circuit board (FPCB).

According to various example embodiments, the wearable electronic device may include a substrate disposed between the first housing and the second housing and including at least one electronic element, wherein the substrate is disposed between at least a portion of the battery and the first housing.

According to various example embodiments, the first molding layer may include a first soft molding layer (e.g., the first soft molding layer 2211 of FIG. 6) disposed between the battery and the substrate, and a second soft molding layer (e.g., the second soft molding layer 2212 of FIG. 6) disposed between the battery and the second molding layer.

According to various example embodiments, the wearable electronic device may include a heat dissipation member comprising a heat dissipating material (e.g., the heat dissipation member 260 of FIG. 6) disposed in the first soft molding layer between the battery and the substrate.

According to various example embodiments, the first molding layer may include a flame-retardant material.

According to various example embodiments, the compression rate may be in a range of 10% to 50%.

According to various example embodiments, the first molding layer may comprise polyurethane foam.

According to various example embodiments, the second molding layer may be comprise epoxy.

According to various example embodiments, the second housing may include an annular opening (e.g., the opening 2201 in FIG. 4) in a center thereof, and the wearable electronic device may be worn on a finger through the opening.

While the disclosure has been illustrated and described with reference to various example embodiments, it will be understood that the various example embodiments are intended to be illustrative, and are not intended to be limiting. It will be further understood by those skilled in the art that various changes or modifications may be made without departing from the true spirit and full scope of the disclosure, including the appended claims and their equivalents. It will also be understood that any of the embodiment(s) described herein may be used in conjunction with any other embodiment(s) described herein.

What is claimed is:

1. A wearable electronic device comprising:
a first housing;
a second housing coupled to an inner circumferential surface of the first housing; and
at least one battery disposed between the first housing and the second housing,
wherein the second housing comprises:
a first molding layer disposed to be at least partially in contact with the at least one battery and having a specified compression rate; and
a second molding layer covering the first molding layer and coupled to the first housing.

2. The wearable electronic device of claim 1, wherein the first molding layer is disposed to not be visible from the outside.

3. The wearable electronic device of claim 1, wherein the first molding layer is disposed to overlap the battery and to cover all of the battery.

4. The wearable electronic device of claim 1, wherein the battery has a curvature corresponding to a curvature of the first housing.

5. The wearable electronic device of claim 1, wherein the battery is attached to at least a portion of the first housing via an adhesive member.

6. The wearable electronic device of claim 1, wherein the first housing and/or the second housing has an annular shape.

7. The wearable electronic device of claim 1, comprising a support member disposed between the battery and the second molding layer.

8. The wearable electronic device of claim 7, wherein the support member includes a plate-shaped metal material disposed inside the first molding layer.

9. The wearable electronic device of claim 7, wherein the support member is disposed between the first molding layer and the second molding layer and is attached to the outer surface of the first molding layer.

10. The wearable electronic device of claim 1, comprising at least one cavity provided between the first molding layer and the second molding layer.

11. The wearable electronic device of claim 1, comprising a substrate disposed between the first housing and the second housing and including at least one electronic element disposed thereon,
wherein the substrate is spaced apart from the battery.

12. The wearable electronic device of claim 11, wherein the substrate comprises a flexible printed circuit board (FPCB).

13. The wearable electronic device of claim 1, comprising a substrate disposed between the first housing and the second housing and comprises at least one electronic element disposed thereon,
wherein the substrate is disposed between at least a portion of the battery and the first housing.

14. The wearable electronic device of claim 13, wherein the first molding layer comprises a first soft molding layer disposed between the battery and the substrate, and a second soft molding layer disposed between the battery and the second housing.

15. The wearable electronic device of claim 14, comprising a heat dissipation member disposed in the first soft molding layer and between the battery and the substrate.

16. The wearable electronic device of claim 1, wherein the first molding layer comprises a flame-retardant material.

17. The wearable electronic device of claim 1, wherein the compression rate is in a range of 10% to 50%.

18. The wearable electronic device of claim 1, wherein the first molding layer comprises polyurethane foam.

19. The wearable electronic device of claim 1, wherein the second molding layer comprises epoxy.

20. The wearable electronic device of claim 1, wherein the second housing comprises an annular opening at a center thereof, and
wherein the wearable electronic device is configured to be worn on a finger through the opening.
